(19) United States
(12) Patent Application Publication
(10) Pub. No.: US 2018/0200261 A1
(43) Pub. Date: Jul. 19, 2018

(54) METHODS AND MATERIALS FOR TREATING CANCER

(71) Applicant: Mayo Foundation for Medical Education and Research, Rochester, MN (US)

(72) Inventors: Min Deng, Rochester, MN (US); Zhenkun Lou, Rochester, MN (US)

(73) Assignee: Mayo Foundation for Medical Education and Research, Rochester, MN (US)

(21) Appl. No.: 15/746,072

(22) PCT Filed: Jul. 13, 2016
(86) PCT No.: PCT/US16/42097

§ 371 Date: Jan. 19, 2018

Related U.S. Application Data
(60) Provisional application No. 62/194,658, filed on Jul. 20, 2015.

(51) Int. Cl. A61K 31/551; A61P 35/00; A61K 31/502

(52) U.S. Cl. CPC A61K 31/551; A61K 31/502; A61P 35/00

(57) ABSTRACT

This document provides methods and materials for treating cancer. For example, methods and materials for using an inhibitor of a BET polypeptide to increase the sensitivity of cancer cells to treatment with an inhibitor of a PARP polypeptide are provided.

### CROSS-REFERENCE TO RELATED APPLICATIONS

This application claims priority to U.S. Application Ser. No. 62/194,658, filed on Jul. 20, 2015. The disclosure of the prior application is considered part of the disclosure of this application, and is incorporated in its entirety into this application.

### BACKGROUND

### 1. Technical Field

This document relates to methods and materials involved in treating cancer. For example, this document provides methods and materials for using an inhibitor of a bromodomain and extraterminal domain (BET) polypeptide to increase the sensitivity of cancer cells to treatment with an inhibitor of a poly ADP ribose polymerase (PARP) polypeptide.

### 2. Background Information

Cancer is the second-leading cause of death in the United States. But survival rates are improving for many types of cancer, thanks to improvements in cancer screening and cancer treatment. Several examples of cancer include breast cancer, ovarian cancer, and osteosarcoma.

Breast cancer is a cancer that develops from breast tissue and is the most common invasive cancer in women. Breast cancer is usually treated with surgery, which may be followed by chemotherapy or radiation therapy, or both chemotherapy and radiation therapy.

Ovarian cancer is a type of cancer that begins in the ovaries. It often goes undetected until it has spread within the pelvis and abdomen. Surgery and chemotherapy are generally used to treat ovarian cancer.

Osteosarcoma is a type of bone cancer that begins in the cells that form your bones. Osteosarcoma is most often found in the long bones in your arms and legs, though it can occur in any bone. Osteosarcoma tends to occur in children and young adults, but it can also occur in older adults. Osteosarcoma treatment often involves surgery, chemotherapy and radiation therapy.

### SUMMARY

This document provides methods and materials for treating cancer. For example, this document provides methods and materials for using an inhibitor of a BET polypeptide to increase the sensitivity of cancer cells to treatment with an inhibitor of a PARP polypeptide. As described herein, an inhibitor of a BET polypeptide can be administered to a mammal (e.g., a human) to increase the sensitivity of the mammal's cancer cells to an inhibitor of a PARP polypeptide. After the sensitivity of the mammal's cancer cells to a PARP polypeptide inhibitor is increased, a PARP polypeptide inhibitor can be administered to the mammal to reduce the number of cancer cells within the mammal.

In general, one aspect of this document features a method for treating cancer in a mammal. The method comprises, or consists essentially of, (a) administering a BET inhibitor to the mammal under conditions wherein the sensitivity of cancer cells within the mammal to a PARP inhibitor is increased, and (b) administering, after at least about 12 hours (e.g., at least about 18, 24, 48, 72, or 96 hours) of administering the BET inhibitor to the mammal, a PARP inhibitor to the mammal under conditions wherein the number of cancer cells within the mammal is reduced. The mammal can be a human. The cancer can be breast cancer, ovarian cancer, or an osteosarcoma. The BET inhibitor can be selected from the group consisting of JQ1, OTX015, i-BET-762, RVX-208, I-BET-762, and MS436. The PARP inhibitor can be selected from the group consisting of AZD2281, ABT-888, BSI-201, BMN673, and AG-14361.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIGS. 1A-B. BET inhibitor affects DNA repair efficiency. (A and B) HeLa cells treated with (JQ1) or without (CK) 250 nM JQ1 were irradiated (2 Gy) and probed with the indicated antibodies at different time points.

FIG. 2. BET inhibitors inhibit homologous recombination. After transfection of I-SceI, HeLa DR-GFP cells were treated with DMSO, JQ1, or OTX015 for 36 hours and subjected to flow cytometry analysis to examine recombination induced by I-SceI digestion.

FIG. 3. BET inhibitors (JQ1, OTX015, and i-BET-762) affect cell sensitivity to PARP inhibitor. OVCAR10 cells were plated in 6 well plates at a density of 500 cells/well. JQ1 (100 nM), OTX015 (100 nM), and i-BET-762 (250 nM) were added together with different concentrations of AZD2281 (0, 0.1, 1, or 10 μM). Controls (CK) did not contain a BET inhibitor. The cells were cultured for 1 to 2 weeks until clones were visible for counting.

FIGS. 4A-C. BET inhibitors affect cell sensitivity to PARP inhibitor in breast cancer cells. Breast cancer cells (BT549, T47D, and MDA231) were plated in 6 well plates with at a density of about 500 cells/well. JQ1 (100 nM) or DMSO (CK) was added together with different concentrations of AZD2281 (0, 0.1, 1, and 10 μM), and the cells were cultured for 1 to 2 weeks until clones were visible for counting.

FIGS. 5A-C. BET inhibitors affect cell sensitivity to PARP inhibitor in ovarian cancer cells. Ovarian cancer cells (OVCAR7, OVCAR8, and OVCAR10) were plated in 6 well plates with at a density of 500 cells/well. JQ1 (100 nM) or DMSO (CK) was added together with different concentrations of AZD2281 (0, 0.1, 1, or 10 μM), and the cells were cultured for 1 to 2 weeks until clones were visible for counting.

FIG. 6. BET inhibitors affect cell sensitivity to PARP inhibitor in osteosarcoma cancer cells. Osteosarcoma cancer cells (U2OS) were plated in 6 well plates with at a density of 500 cells/well. JQ1 (100 nM) or DMSO (CK) was added together with different concentrations of AZD2281 (0, 0.1, 1, or 10 μM), and the cells were cultured for 1 to 2 weeks until clones were visible for counting.

### DETAILED DESCRIPTION

This document provides methods and materials for treating cancer. For example, this document provides methods and materials for using an inhibitor of a BET polypeptide to increase the sensitivity of cancer cells to treatment with an inhibitor of a PARP polypeptide. Once the sensitivity of the mammal's cancer cells to a PARP polypeptide inhibitor is increased, a PARP polypeptide inhibitor can be administered to the mammal to reduce the number of cancer cells within the mammal.

Any type of mammal having cancer can be treated as described herein. For example, humans and other primates such as monkeys having cancer can be treated with one or more inhibitors of a BET polypeptide to increase the sensitivity of cancer cells to treatment with an inhibitor of a PARP polypeptide followed by treatment with one or more PARP polypeptide inhibitors to reduce the number of cancer cells present within the mammal. In some cases, dogs, cats, horses, cows, pigs, sheep, mice, and rats can be treated with one or more BET polypeptide inhibitors followed by one or more PARP polypeptide inhibitors as described herein.

Any appropriate cancer can be treated as described herein. For example, breast cancer, ovarian cancer, osteosarcoma, lung cancer, prostate cancer, liver cancer, pancreatic cancer, brain/CNS tumors, and colon, rectal, or colorectal cancer can be treated with one or more BET polypeptide inhibitors followed by one or more PARP polypeptide inhibitors as described herein.

Any appropriate method can be used to identify a mammal having cancer. For example, imaging techniques and biopsy techniques can be used to identify mammals (e.g., humans) having cancer.

Once identified as having cancer, the mammal can be administered or instructed to self-administer one or more BET polypeptide inhibitors to increase the sensitivity of cancer cells to treatment with an inhibitor of a PARP polypeptide. Examples of BET polypeptide inhibitors include, without limitation, JQ1 ((S)-tert-butyl 2-(4-(4-chlorophenyl)-2,3,9-trimethyl-6H-thieno[3,2-f][1,2,4]triazolo[4,3-a][1,4]diazepin-6-yl)acetate), OTX015 ((6S)-4-(4-chlorophenyl)-N-(4-hydroxyphenyl)-2,3,9-trimethyl-6H-thieno[3,2-f][1,2,4]triazolo[4,3-a][1,4]diazepine-6-acetamide), i-BET-762 ((S)-2-(6-(4-chlorophenyl)-8-methoxy-1-methyl-4H-benzo[f][1,2,4]triazolo[4,3-a][1,4]diazepin-4-yl)-N-ethylacetamide), RVX-208 (2-[4-(2-Hydroxyethoxy)-3,5-dimethylphenyl]-5,7-dimethoxy-4(3H)-quinazolinone), I-BET-762 (2-[(4S)-6-(4-chlorophenyl)-8-methoxy-1-methyl-4H-[1,2,4]triazolo[4,3-a][1,4]benzodiazepin-4-yl]-N-ethylacetamide), and MS436 ((E)-4-[2-(2-Amino-4-hydroxy-5-methylphenyl)diazenyl]-N-2-pyridinylbenzenesulfonamide). In some cases, two or more BET polypeptide inhibitors (e.g., two, three, four, five, or more BET polypeptide inhibitors) can be administered to a mammal to increase the sensitivity of cancer cells to treatment with an inhibitor of a PARP polypeptide.

In some cases, one or more BET polypeptide inhibitors can be formulated into a pharmaceutically acceptable composition for administration to a mammal having cancer. For example, a therapeutically effective amount of a BET polypeptide inhibitor (e.g., JQ1, OTX015, or i-BET-762) can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules.

Once the mammal is treated with a BET polypeptide inhibitor to increase the sensitivity of cancer cells to treatment with an inhibitor of a PARP polypeptide, then a PARP polypeptide inhibitor can be administered to the mammal to reduce the number of cancer cells present within the mammal. Examples of PARP polypeptide inhibitors include, without limitation, AZD2281 (4-[(3-[(4-cyclopropylcarbonyl)piperazin-4-yl]carbonyl)-4-fluorophenyl]methyl(2H)phthalazin-1-one), ABT-888 (2-((R)-2-Methylpyrrolidin-2-yl)-1H-benzimidazole-4-carboxamide), BSI-201 (4-iodo-3-nitrobenzamide), BMN673 (talazoparib), and AG-14361 (1-(4-((dimethyl-amino)methyl)phenyl)-8,9-dihydro-2,7,9a-triazabenzo[cd]azulen-6(7H)-one). In some cases, two or more PARP polypeptide inhibitors (e.g., two, three, four, five, or more BET polypeptide inhibitors) can be administered to a mammal to reduce the number of cancer cells present within the mammal.

In some cases, one or more BET polypeptide inhibitors can be administered to a mammal once or multiple times over a period of time ranging from days to weeks. During this period of time, the mammal can be treated in a manner that does not include the administration of any PARP polypeptide inhibitors. At least 12 hours after the final treatment with a BET polypeptide inhibitor, the mammal can be treated with one or more PARP polypeptide inhibitors.

In some cases, one or more PARP polypeptide inhibitors can be formulated into a pharmaceutically acceptable composition for administration to a mammal having cancer. For example, a therapeutically effective amount of a PARP polypeptide inhibitor (e.g., JQ1, OTX015, or i-BET-762) can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules.

Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition containing one or more BET polypeptide inhibitors or one or more PARP polypeptide inhibitors can be designed for oral or parenteral (including subcutaneous, intramuscular, intravenous, and intradermal) administration. When being administered orally, a pharmaceutical composition can be in the form of a pill, tablet, or capsule. Compositions suitable for parenteral administration include aqueous and non-aqueous sterile injection solutions that can contain anti-oxidants, buffers, bacteriostats, and solutes that render the formulation isotonic with the blood of the intended recipient. The formulations can be presented in unit-dose or multi-dose containers, for example, sealed ampules and vials, and may be stored in a freeze dried (lyophilized) condition requiring only the addition of the sterile liquid carrier, for example, water for injections, immediately prior to use. Extemporaneous injection solutions and suspensions may be prepared from sterile powders, granules, and tablets.

In some cases, a pharmaceutically acceptable composition including one or more BET polypeptide inhibitors or one or more PARP polypeptide inhibitors can be administered locally or systemically. For example, a composition provided herein can be administered locally by injection into tumors. In some cases, a composition provided herein can be administered systemically, orally, or by injection to a mammal (e.g., a human).

Effective doses can vary depending on the severity of the cancer, the route of administration, the age and general health condition of the subject, excipient usage, the possibility of co-usage with other therapeutic treatments such as use of other agents, and the judgment of the treating physician.

An effective amount of a composition containing one or more BET polypeptide inhibitors can be any amount that increases the sensitivity of cancer cells to treatment with an inhibitor of a PARP polypeptide without producing significant toxicity to the mammal. For example, an effective amount of a BET polypeptide inhibitor such as JQ1 can be from about 50 mg/kg to about 200 mg/kg (e.g., from about 75 mg/kg to about 200 mg/kg, from about 100 mg/kg to about 200 mg/kg, from about 50 mg/kg to about 150 mg/kg, or from about 50 mg/kg to about 100 mg/kg). In some cases, between about 4 g and about 16 g of a BET polypeptide inhibitor can be administered to an average sized human (e.g., about 75-85 kg human) daily for about 3 to about 12 weeks (e.g., about 3 to 10 weeks, about 3 to 8 weeks, about 3 to 6 weeks, about 3 to 4 weeks).

An effective amount of a composition containing one or more PARP polypeptide inhibitors can be any amount that reduces the number of cancer cells present within the mammal without producing significant toxicity to the mammal. For example, an effective amount of a PARP polypeptide inhibitor such as AZD2281 can be from about 50 mg/kg to about 200 mg/kg (e.g., from about 75 mg/kg to about 200 mg/kg, from about 100 mg/kg to about 200 mg/kg, from about 50 mg/kg to about 150 mg/kg, or from about 50 mg/kg to about 100 mg/kg). In some cases, between about 4 g and about 16 g of a BET polypeptide inhibitor can be administered to an average sized human (e.g., about 75-85 kg human) daily for about 3 to about 12 weeks (e.g., about 3 to 10 weeks, about 3 to 8 weeks, about 3 to 6 weeks, about 3 to 4 weeks).

If a particular mammal fails to respond to a particular amount, then the amount of BET polypeptide inhibitor and/or PARP polypeptide inhibitor can be increased by, for example, two fold. After receiving this higher amount, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., cancer) may require an increase or decrease in the actual effective amount administered.

The frequency of administration of a BET polypeptide inhibitor can be any amount that increases the sensitivity of cancer cells to treatment with an inhibitor of a PARP polypeptide without producing significant toxicity to the mammal. For example, the frequency of administration of a BET polypeptide inhibitor can be from about once a week to about eight times a month.

The frequency of administration of a PARP polypeptide inhibitor can be any amount that reduces the number of cancer cells present within the mammal without producing significant toxicity to the mammal. For example, the frequency of administration of a PARP polypeptide inhibitor can be from about once a week to about eight times a month.

The frequency of administration of a BET polypeptide inhibitor and/or a PARP polypeptide inhibitor can remain constant or can be variable during the duration of treatment. A course of treatment with a composition containing a BET polypeptide inhibitor and/or a PARP polypeptide inhibitor can include rest periods. For example, a composition containing one or more BET polypeptide inhibitors can be administered daily over a two week period followed by a two week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., cancer) may require an increase or decrease in administration frequency.

An effective duration for administering a composition containing one or more BET polypeptide inhibitors can be any duration that increases the sensitivity of cancer cells to treatment with an inhibitor of a PARP polypeptide without producing significant toxicity to the mammal. In some cases, the effective duration can vary from several days to several weeks. In general, the effective duration for increasing the sensitivity of cancer cells to treatment with an inhibitor of a PARP polypeptide can range in duration from about one week to about 12 weeks.

An effective duration for administering a composition containing one or more PARP polypeptide inhibitors can be any duration that reduces the number of cancer cells present within the mammal without producing significant toxicity to the mammal. In some cases, the effective duration can vary from several days to several weeks. In general, the effective duration for reducing the number of cancer cells present within the mammal can range in duration from about three weeks to about 20 weeks.

Multiple factors can influence the actual effective duration used for a particular treatment. For example, an effective duration can vary with the frequency of administration, effective amount, use of multiple treatment agents, route of administration, and severity of the condition being treated.

In certain instances, a course of treatment, the number of cancer cells present within a mammal, and/or the severity of one or more symptoms related to the condition being treated (e.g., cancer) can be monitored. Any appropriate method can be used to determine whether or not the number of cancer cells present within a mammal is reduced. For example, imaging techniques can be used to assess the number of cancer cells present within a mammal.

The invention will be further described in the following examples, which do not limit the scope of the invention described in the claims.

### EXAMPLES

### Example 1—Increasing the Sensitivity of Cancer Cells to Treatment with PARP Polypeptide Inhibitors

### Cell Lines

U2OS cells were cultured in Mycoy's 5A with 10% FBS. HeLa, OVCAR7, OVCAR8, OVCAR10, BT549, and T47D cells were cultured in DMEM with 10% FBS. MDA231 cells were cultured in ATCC-formulated Leibovitz's L-15 medium.

### Chemicals

(+)-JQ1, OTX015, I-BET-762, and AZD2281 were purchased from Selleck Chemicals Inc. All compounds were dissolved in DMSO to form a stock solution of 100 μM.

### Immunofluorescence

To visualize ionizing radiation induced foci (IRIF), cells were cultured on coverslips and treated with 5 Gy ionizing radiation (IR) followed by recovery as indicated. Cells were then washed in PBS, incubated in 3% paraformaldehyde for 15 minutes, and permeabilized in 0.5% Triton solution for 5 minutes at room temperature. Samples were blocked with 5% goat serum and then incubated with primary antibodies for 30 minutes. Samples were washed three times and incubated with secondary antibodies for 30 minutes. Cells were then stained with DAPI to visualize nuclear DNA. The coverslips were mounted onto glass slides with anti-fade solution and visualized using a Nikon eclipse 80i fluorescence microscope. The following antibodies were used in immunofluorescence: rH2Ax (Millipore) and Rad51 (GeneTex).

HR Assay

A HeLa clone with the integrated HR reporter DR-GFP was obtained from Dr. Maria Jasin (Sloan Kettering). HeLa-DR-GFP cells were transfected with an I-SceI expression vector (pCBA-I-SceI). 18 hours after transfection, cells were treated with DMSO or 250 nM of JQ1 for 36 hours and subjected to flow cytometric analysis to examine recombination induced by I-SceI digestion. Results are presented as a percentage of control treatment.

### PARP Inhibitor Sensitivity Assay

Cells were plated in 6 well plates with at a density of 500 cells/well. BET inhibitors were added together with different concentrations of AZD2281 (0, 0.1, 1, or 10 μM), and the cells were cultured for 1 to 2 weeks until the colonies were visible for counting.

### BET Inhibitor Affects DNA Repair Efficiency

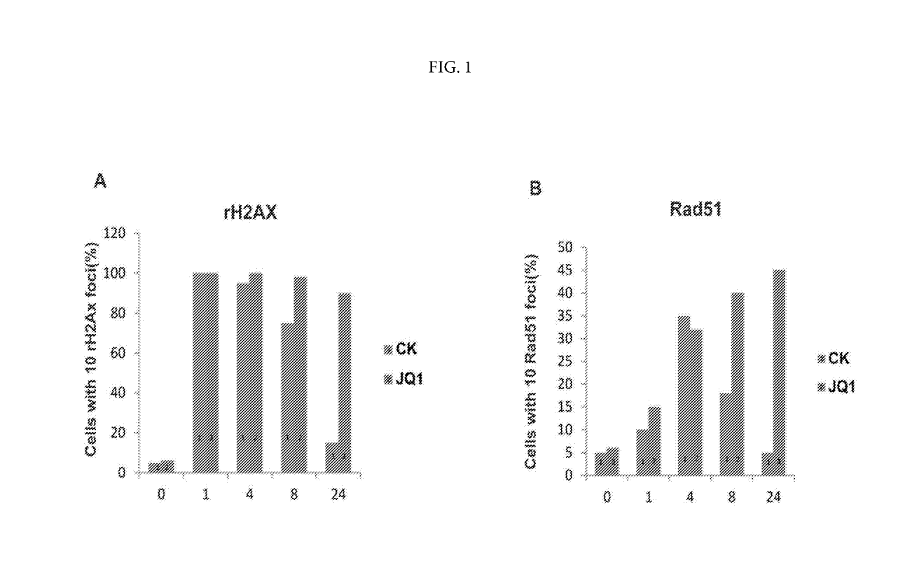

To check the effect of BET inhibitor in the DNA repair pathway, the foci of rH2Ax and RAD51 were examined (FIG. 1). rH2Ax is the marker of damaged DNA. RAD51 is an enzyme involved in homology recombination. Both proteins can form foci at damaged DNA sites.

In the control group, the percentage of rH2Ax positive cells gradually decreased to about 15% 24 hours after IR. In JQ1 treated cells, however, the rH2Ax foci sustained at high levels even at 24 hours after IR, indicating un-repaired DNA damage in the cells. JQ1 also affected RAD51 in a similar manner. These results demonstrate that inhibition of BET impaired the DNA repair pathway.

### BET Inhibitors Inhibit Homologous Recombination

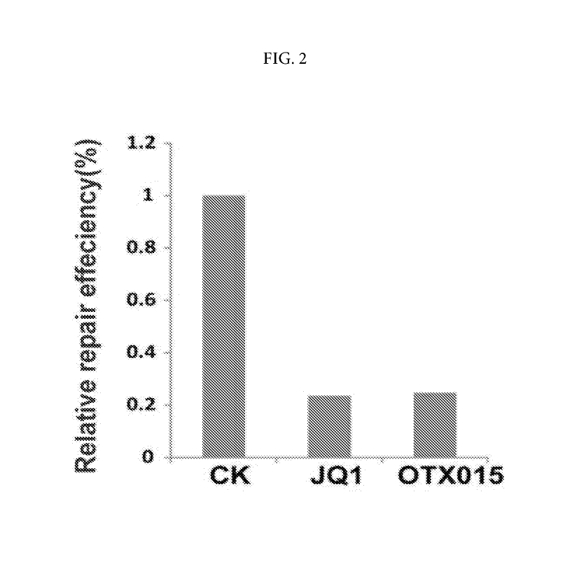

To check the effect of BET inhibitors on homologous recombination, the Hela DR-GFP reporter system was utilized to monitor the ratio of homologous recombination after treatment with BET inhibitors. BET inhibitors decreased the homologous recombination efficiency (FIG. 2).

### BET Inhibitors Affect Cell Sensitivity to PARP Inhibitor in Breast Cancer Cells

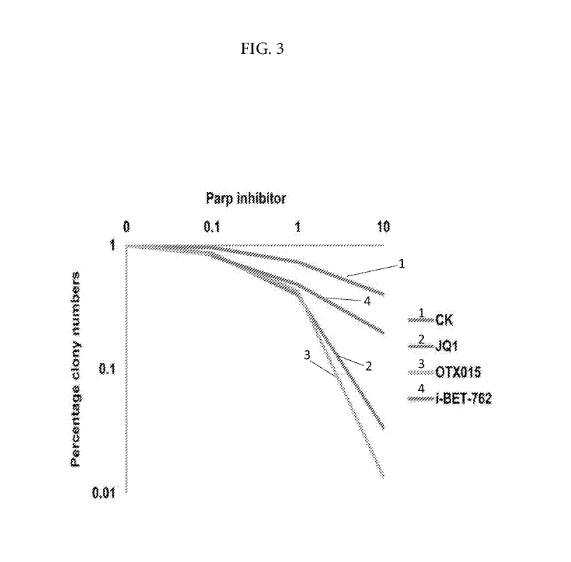

To check whether BET inhibitors affect cell sensitivity to PARP inhibitor, cells were treated with BET inhibitors together with a PARP inhibitor. All three BET inhibitors tested sensitized the cells to a PARP inhibitor (FIG. 3).

### BET Inhibitors Affect Cell Sensitivity to PARP Inhibitor in Cancer Cells

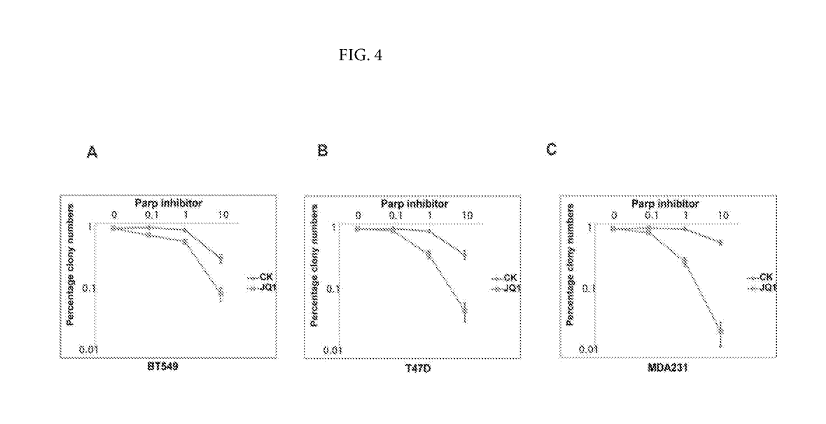

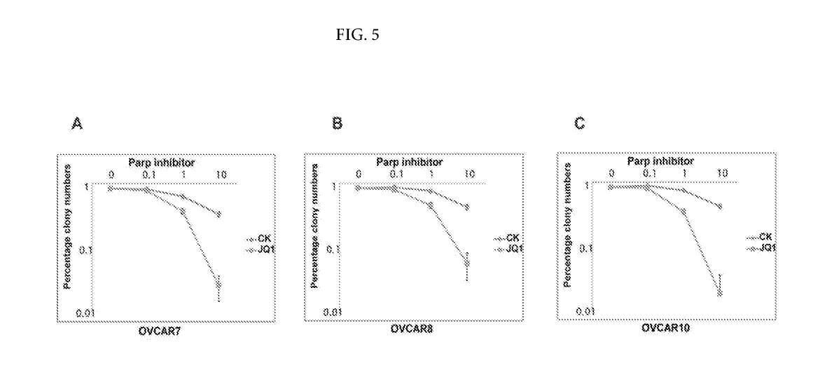

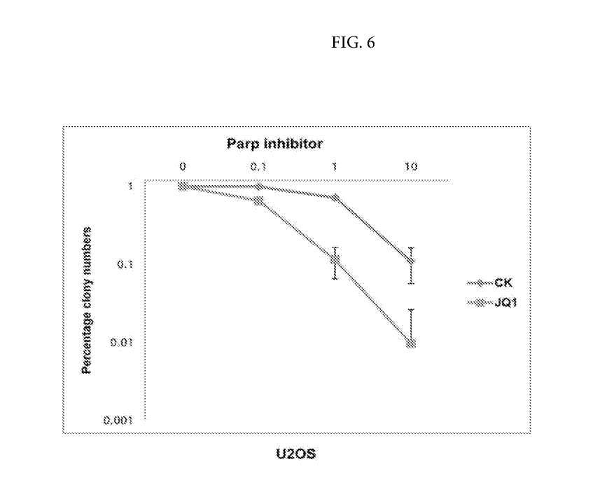

The effect of BET inhibitors were assessed in three different breast cancer cell lines (BT549, T47D, and MDA231), three different ovarian cancer cell lines (OVCAR7, OVCAR8, and OVCAR10), and an osteosarcoma cancer cell line (U2OS). In all these cell lines, BET inhibitors increased cell sensitivity to AZD2281, a PARP inhibitor (FIGS. 4-6).

### Example 2—Treating Cancer

A human having cancer (e.g., breast cancer, ovarian cancer, or an osteosarcoma) is identified as having cancer. Once identified, the human is treated with a BET inhibitor (e.g., JQ1, OTX015, or i-BET-762) to increase the sensitivity of the human's cancer cells to treatment with a PARP inhibitor. After being administered the BET inhibitor, a PARP inhibitor (e.g., AZD2281) is administered to the human. Briefly, a BET inhibitor (e.g., JQ1, OTX015, or i-BET-762) is administered to the human at least once every day for at least about one week without any treatments with a PARP inhibitor. After the at least about 3 to 4 days without any treatments with a PARP inhibitor, a PARP inhibitor (e.g., ABT2281) is administered to the human.

In some cases, a BET inhibitor (e.g., JQ1, OTX015, or i-BET-762) is administered to the human without administering a PARP inhibitor. At least about 24 hours after administration of the BET inhibitor, a PARP inhibitor (e.g., AZD2281) is administered to the human.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating cancer in a mammal, wherein said method comprises:
(a) administering a BET inhibitor to said mammal under conditions wherein the sensitivity of cancer cells within said mammal to a PARP inhibitor is increased, and
(b) administering, after at least about 12 hours of administering said BET inhibitor to said mammal, a PARP inhibitor to said mammal under conditions wherein the number of cancer cells within said mammal is reduced.

2. The method of claim 1, wherein said mammal is a human.

3. The method of claim 1, wherein said cancer is breast cancer, ovarian cancer, or an osteosarcoma.

4. The method of claim 1, wherein said BET inhibitor is selected from the group consisting of JQ1, OTX015, i-BET-762, RVX-208, I-BET-762, and MS436.

5. The method of claim 1, wherein said PARP inhibitor is selected from the group consisting of AZD2281, ABT-888, BSI-201, BMN673, and AG-14361.
